(19) United States
(12) Patent Application Publication
(10) Pub. No.: US 2025/0151993 A1
(43) Pub. Date: May 15, 2025

(54) MULTIPLE BOTTLE LIQUID FEED SYSTEM

(71) Applicant: Boston Scientific Scimed, Inc., Maple Grove, MN (US)

(72) Inventors: Scott Edward Corbeil, Litchfield, NH (US); Ryan V. Wales, Northborough, MA (US); Aakash Deora, Marlborough, MA (US)

(73) Assignee: Boston Scientific Scimed, Inc., Maple Grove, MN (US)

(21) Appl. No.: 18/944,291

(22) Filed: Nov. 12, 2024

Related U.S. Application Data
(60) Provisional application No. 63/599,251, filed on Nov. 15, 2023.

(51) Int. Cl. A61B 1/015

(52) U.S. Cl. CPC A61B 1/015

(57) ABSTRACT

Devices, systems, and methods for a fluid supply for an endoscope. A plurality of bottles each include an outlet tube with a float valve; they are connected in series along a connection line. When each bottle is depleted, its float valve closes, allowing the remaining bottles to be drawn from.

### CROSS-REFERENCE TO RELATED APPLICATIONS

This application claims the benefit of U.S. Provisional Patent Application Ser. No. 63/599,251 filed on Nov. 15, 2023, the disclosure of which is incorporated herein by reference.

### FIELD

This disclosure relates generally to valve assemblies and methods, and particularly to liquid supply during endoscopy.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a liquid feed system includes a connector line in fluid communication with a pump and a plurality of bottles, each bottle having an outlet tube with a float valve. The connector line is in fluid communication with each of the outlet tubes in series, such that upon activation of the pump, fluid is drawn from the plurality bottles. When a liquid supply available in any one of the plurality of bottles is depleted, the float valve associated with the depleted bottle closes to prevent further draw from the depleted bottle.

Alternatively or additionally to any of the examples above, each float valve can be integral with its associated outlet tube.

Alternatively or additionally to any of the examples above, each float valve can include a housing, the housing having an upper end containing a stopper when the float valve is open and a lower end seating the stopper when the float valve is closed.

Alternatively or additionally to any of the examples above, each float valve can include one or more openings in the housing between the upper end and the lower end.

Alternatively or additionally to any of the examples above, each float valve can include a plurality of openings in the housing between the upper end and the lower end.

Alternatively or additionally to any of the examples above, each float valve can include a spherical stopper.

Alternatively or additionally to any of the examples above, each float valve can include a tapering valve seat and a stopper having a tapering end matching the tapering valve seat.

Alternatively or additionally to any of the examples above, each float valve can include an annular stopper positioned exterior to outlet tube.

Alternatively or additionally to any of the examples above, each float valve can include one or more openings in the outlet tube positioned such that the annular stopper slides along the surface of the outlet tube to seal the openings when the float valve is closed.

As another example, a liquid dispenser includes a cap sized to receive a bottle in an inverted position; a float valve positioned to extend into a bottle when the bottle is received by the cap in an inverted position, the float valve moving between an open position allowing liquid to enter from the bottle and a closed position when the bottle is depleted; and a connector in fluid communication with the float valve.

Alternatively or additionally to any of the examples above, the liquid dispenser can further include legs extending below the connector to allow the liquid dispenser to stand when connected with an inverted bottle.

Alternatively or additionally to any of the examples above, the cap can be threaded to match the threads of a bottle.

Alternatively or additionally to any of the examples above, the float valve can include a housing having an upper end containing a stopper when the float valve is open and a lower end seating the stopper when the float valve is closed.

Alternatively or additionally to any of the examples above, the float valve can include an opening extending further downward than the stopper when the float valve is in the open position.

As another example, a liquid feed system includes a connector line in fluid communication with a pump; a plurality of bottles; and a plurality of liquid dispensers of any of the examples above, each liquid dispenser receiving, in an inverted position, one of the plurality of bottles. The connector line is in fluid communication with each of the dispensers in series, such that upon activation of the pump, fluid is drawn from the plurality bottles. When a liquid supply available in any one of the plurality of bottles is depleted, the float valve of the liquid dispenser receiving the depleted bottle closes to prevent further draw from the depleted bottle.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3A depicts a schematic view of an illustrative liquid feed system;

FIG. 3B depicts a diagrammatic view of the illustrative liquid feed system connected to components of an illustrative endoscope system;

FIGS. 4A and 4B depict a cross-section side views of an illustrative outlet tube with float valve;

FIGS. 5A and 5B depict a cross-section side views of an illustrative outlet tube with float valve;

FIGS. 6A and 6B depict a cross-section side views of an illustrative outlet tube with float valve;

FIGS. 7A and 7B depict a cross-section side views of an illustrative outlet tube with float valve;

FIGS. 8A and 8B depict a cross-section side views of an illustrative outlet tube with float valve;

FIGS. 9A and 9B depict a cross-section side views of an illustrative outlet tube with float valve;

FIGS. 10A and 10B depict a cross-section side views of an illustrative outlet tube with float valve;

FIG. 11A depicts a perspective view of an illustrative liquid dispenser; and

FIG. 11B depicts a cross-section side view of the liquid dispenser of FIG. 11A.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

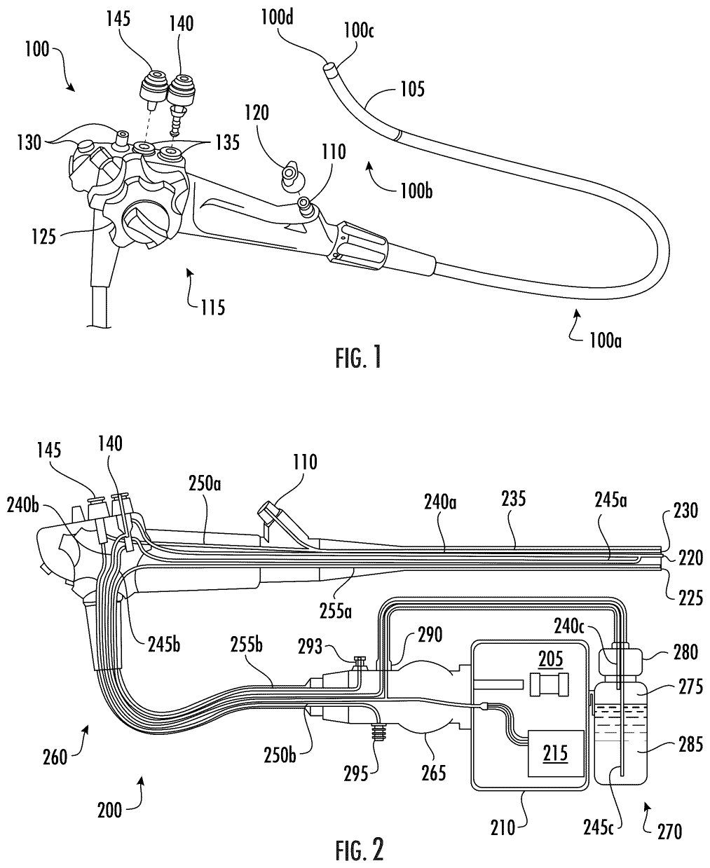

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serve as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir is fluidly connected to the endoscope 100 through the connector portion 265 and the umbilical 260. The gas feed line 240b from the umbilical 260 branches in the connector portion 265 to fluidly communicate with a length of gas supply tubing 240c at the detachable gas/lens wash connection 290, as well as the air pump 215. A length of lens wash tubing 245c runs to the same detachable connection 290 as the gas supply tubing 240c on the connector portion 265. In other embodiments, the connections may be separate and/or separated from each other. The connector portion 265 may also have a detachable irrigation connection 293 for irrigation supply tubing running from a source of irrigation water to the irrigation feed line 255b in the umbilical 260. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

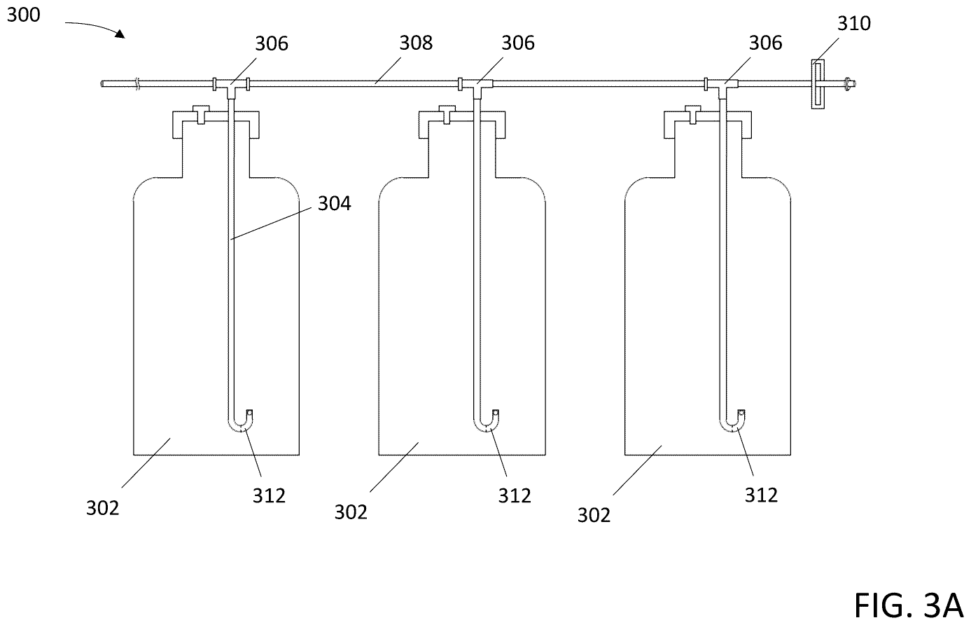

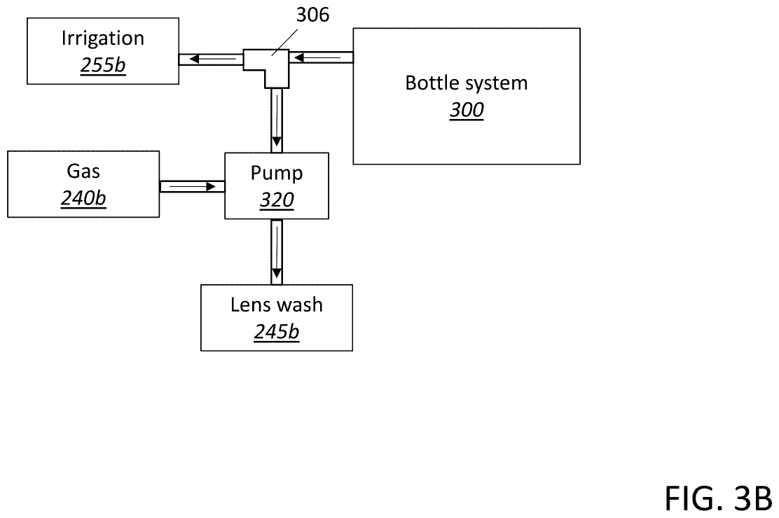

Both the lens wash feed line and the irrigation feed line may be fed from a liquid feed system 300 as illustrated in FIGS. 3A and 3B. The system 300 includes multiple irrigation bottles 302, each of which is connected to a tube system means of an outlet tube 304. The outlet tubes 304 are connected by junctions 306 to a connector line 308, in fluid communication with one or both feed lines at one end. As shown, a clamp 310 may shut off the unused end of the opposite line from the last deployed bottle. Due to the pressure differential along the feed system 300, more liquid will be drawn from the first bottle in the line than from each subsequent bottle. As such, the bottle closest to the outlet will be depleted first.

One of ordinary skill will recognize that the term “depleted” is not necessarily absolute. Some amount of liquid may remain in a bottle that is not readily available for supply to a feed, either due to is position in a bottle (such as below a level accessed by the outlet tube), a lack pressure within a container, or based on an assessment that a particular fluid, due to its concentration of solutes or other issues, is no longer suitable for dispensing. Therefore, in the context of this disclosure, a “depleted” bottle has substantially reduced liquid relative to its initial quantity, such that it should no longer be included in the supply to the feed line. As a non-limited example, a bottle where the remaining liquid is below the openings in the outlet tube permitting liquid ingress is a depleted bottle.

The irrigation feed line 255b may extend directly from the connector line 308 as shown. In order to provide the pressurized liquid typically used in the lens wash feed line, a pump 320 is added as shown in FIG. 3B. A junction 306 splits the output of the bottle system between the irrigation feed line 255b and the pump 320, which also receives gas from the gas feed line 240b. Pressurized liquid is pumped into the lens wash feed line 245b. One of ordinary skill will recognize that, in some embodiments, only one of the irrigation feed line 255b or the lens wash feed line 245b may be supplied by the bottle system 300.

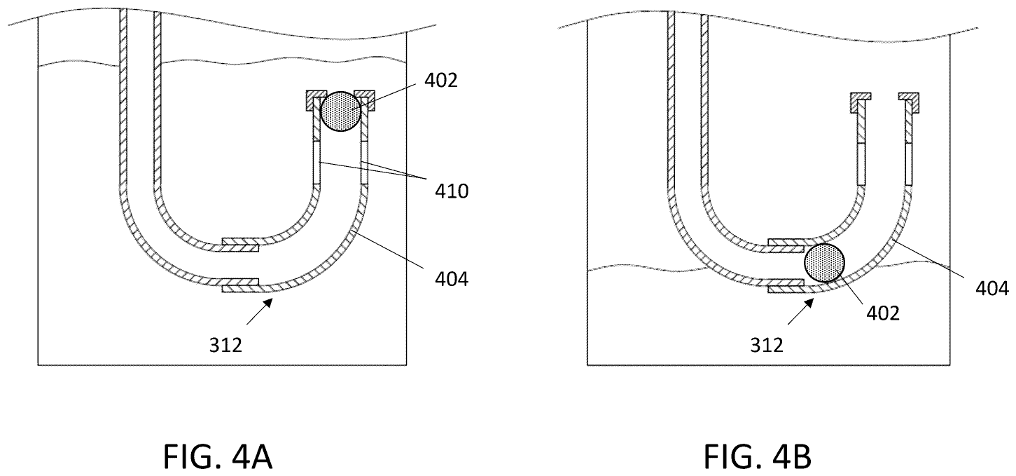

Each outlet tube 304 includes a float valve 312 at its lower end, shown in more detail in FIGS. 4A and 4B. A stopper 402 sits within a float housing 404 that is open to the irrigation liquid in the bottle 302. While the liquid level is above the float valve 312, the position of the stopper 402 allows the water to flow into the outlet tube though openings 410 for supply to the feed line, as shown in FIG. 4A.

FIG. 4B shows that when the bottle is depleted, the liquid level in the bottle 302 falls below the level of the float valve 312, and the stopper 402 becomes seated against the lower portion of the float housing 404, closing the float valve 312. Once the outlet tube 304 of a bottle is sealed in this way, further pressure will instead draw liquid from other bottles within the system 300.

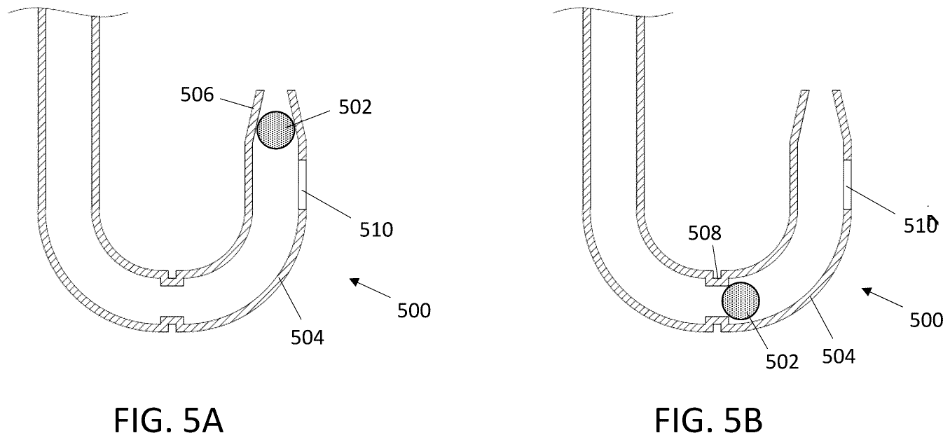

Alternative forms of a float valve are illustrated in FIGS. 5-10. It will be understood by one of ordinary skill that other shapes, sizes, and configurations of float valve are possible, and the specifics described here are not intended to be limiting on the nature and configuration of valving that may be used with the outlet tubes as described.

FIGS. 5A and 5B show a float valve 500 that is integrally molded with the end of the outlet tube 304. The stopper 502 is contained within the float housing 504 by a tapered end portion 506 at the end of the tube, as shown in FIG. 5A. Liquid enters through an opening 510, which is below the level of the stopper 502 when the valve 500 is open. When the bottle is depleted, the stopper 502 moves to a constriction in the tube that acts as a valve seat 508.

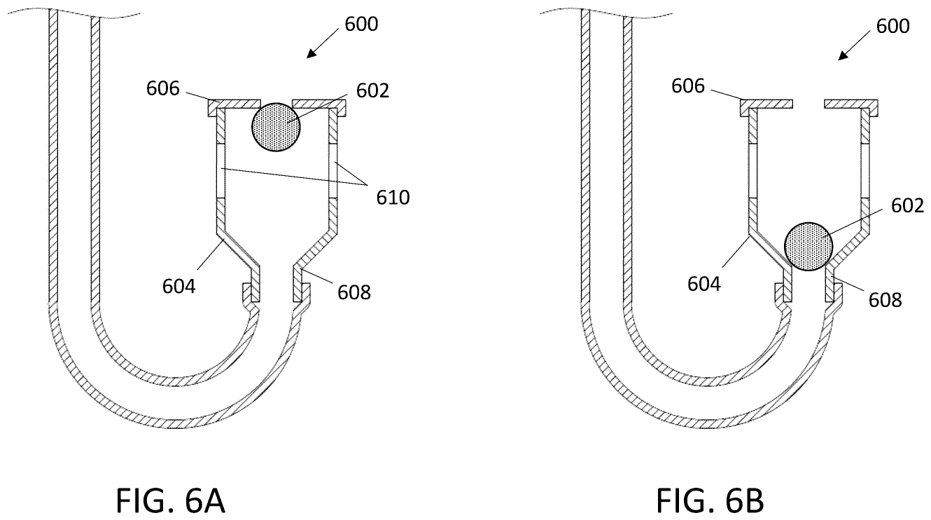

FIGS. 6A and 6B show a float valve 600 in which the housing 604 is connected to the end of the outlet tube. Here a spherical stopper 602 is contained by the upper portion 606 of the housing 604, allowing liquid ingress via the openings 610. When the bottle is depleted, the stopper 602 is seated at the lower constricted portion 608, closing the valve 600.

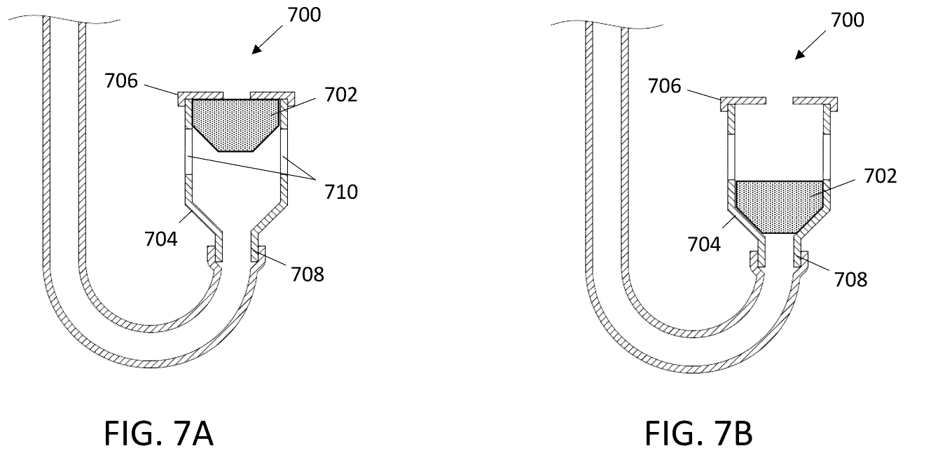

FIGS. 7A and 7B show a float valve 700 with a housing 704 shaped the same as housing 604 described above, but with a stopper 702 sized to occupy the entire width of the housing 704, both at the top portion 706 when the valve 700 is open, and at the valve seat 708 when the valve 700 is closed. The stopper 702 is above the openings 710 when the valve 700 is open. When the bottle is depleted and the stopper 702 moves down, as shown in FIG. 7B, further fluid entering the openings 710 is stopped from entering the outlet tube.

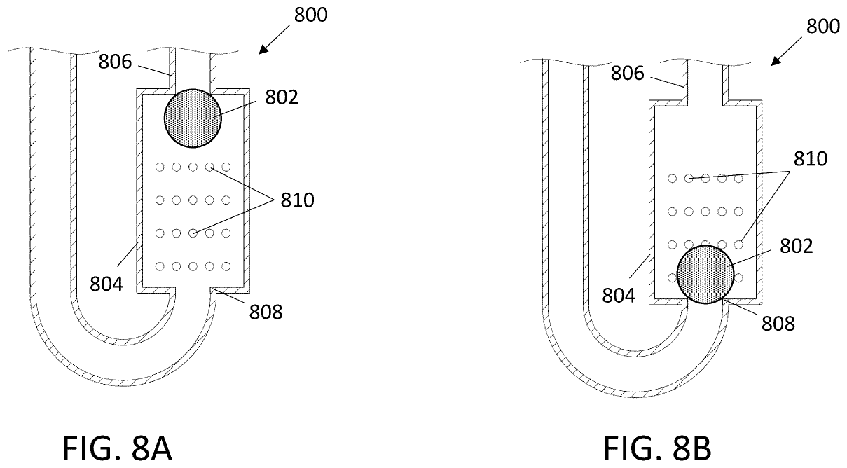

FIGS. 8A and 8B show a float valve 800 with a spherical stopper 802 contained in a symmetrical housing 804. The housing contains channels in both the upper portion 806 and lower portion 808 that are too narrow for the stopper 802 to get through, containing the stopper 802 when the valve 800 is open and causing the stopper 802 to seat against the lower portion 808 when the valve 800 is closed. Small openings 810 in the housing allow liquid ingress until the bottle is depleted.

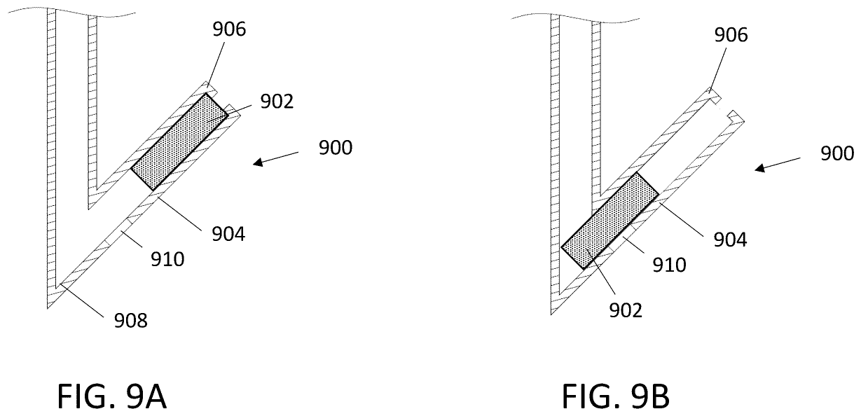

FIGS. 9A and 9B show a float valve 900 in which a sharp corner 908 is added to the tube to form the housing 904 integral with the tube. Here, the stopper 902 is contained between the end 906 and the corner 908, and the stopper blocks the opening 910 when the bottle is depleted.

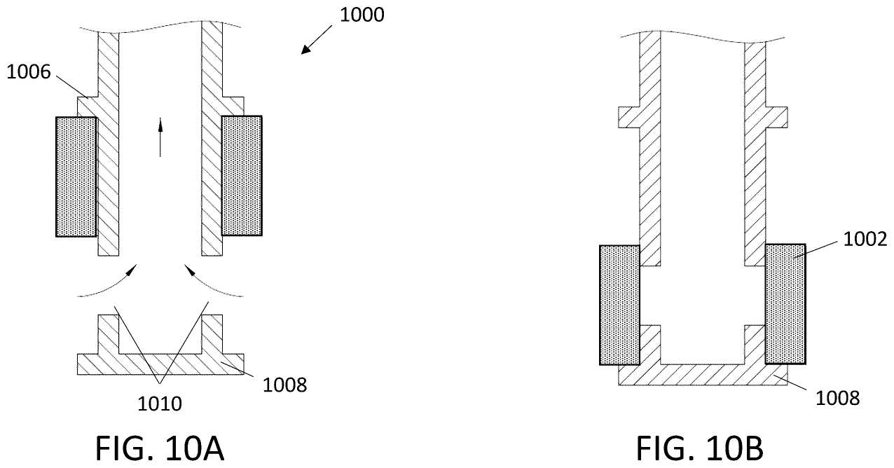

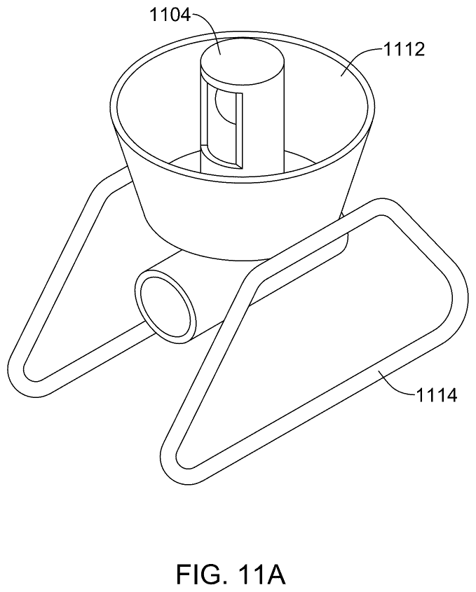

FIGS. 10A and 10B show a float valve 1000 formed by adding flanges 1006 and 1008 to the outside of the tube. The stopper 1002 is annular and surrounds the outside surface of the tube, sliding between an upper flange 1006 and lower flange 1008. Openings 1010 are positioned near the lower flange 1008 so that they are blocked by the stopper 1002 when depleted, as shown in FIG. 10B.

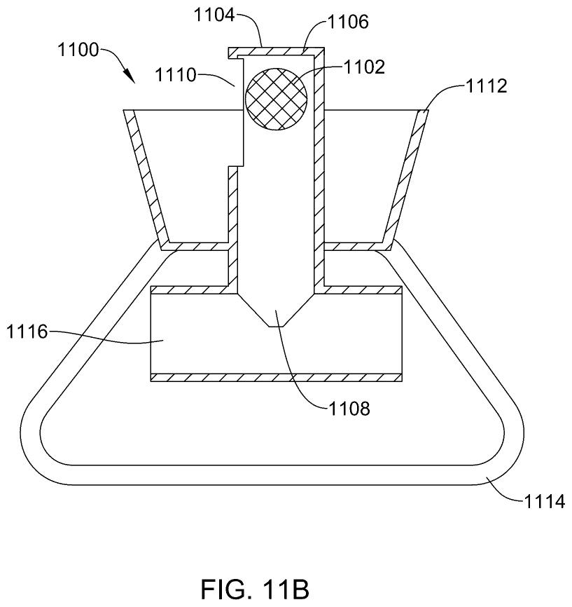

FIGS. 11A and 11B show a dispenser 1100 for an inverted bottle. The stopper 1102 is placed within the housing 1104, moving between the upper portion 1106 and the valve seat 1108. An opening 1110 in the housing 1104 is large enough to allow for ingress of liquid past the stopper 1102. When the bottle is depleted, flow through the dispenser 1100 is stopped by the presence of the 1102 against the seat 1108. The inverted bottle is connected to a cap 1112 surrounding the housing 1104. Legs 1114 allow the bottle and dispenser to stand upright and provide enough space for attachment of the connector line. A connector 1116 extends in both directions from the valve seat 1108, facilitating connection with other bottles to form the liquid feed system.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A liquid feed system, comprising:
a connector line in fluid communication with a pump; and
a plurality of bottles, each bottle having an outlet tube with a float valve;
wherein the connector line is in fluid communication with each of the outlet tubes in series, such that upon activation of the pump, fluid is drawn from the plurality bottles; and
wherein, when a liquid supply available in any one of the plurality of bottles is depleted, the float valve associated with the depleted bottle closes to prevent further draw from the depleted bottle.

2. The liquid feed system of claim 1, wherein each float valve is integral with its associated outlet tube.

3. The liquid feed system of claim 1, wherein each float valve comprises a housing, the housing having an upper end containing a stopper when the float valve is open and a lower end seating the stopper when the float valve is closed.

4. The liquid feed system of claim 3, wherein each float valve comprises one or more openings in the housing between the upper end and the lower end.

5. The liquid feed system of claim 4, wherein each float valve comprises a plurality of openings in the housing between the upper end and the lower end.

6. The liquid feed system of claim 1, wherein each float valve comprises a spherical stopper.

7. The liquid feed system of claim 1, wherein each float valve comprises a tapering valve seat and a stopper having a tapering end matching the tapering valve seat.

8. The liquid feed system of claim 1, wherein each float valve comprises an annular stopper positioned exterior to outlet tube.

9. The liquid feed system of claim 8, wherein each float valve comprises one or more openings in the outlet tube positioned such that the annular stopper slides along the surface of the outlet tube to seal the openings when the float valve is closed.

10. A liquid dispenser, comprising:
a cap sized to receive a bottle in an inverted position;
a float valve positioned to extend into a bottle when the bottle is received by the cap in an inverted position, the float valve moving between an open position allowing liquid to enter from the bottle and a closed position when the bottle is depleted; and
a connector in fluid communication with the float valve.

11. The liquid dispenser of claim 10, further comprising legs extending below the connector to allow the liquid dispenser to stand when connected with an inverted bottle.

12. The liquid dispenser of claim 10, wherein the cap is threaded to match the threads of a bottle.

13. The liquid dispenser of claim 10, wherein the float valve comprises a housing, the housing having an upper end containing a stopper when the float valve is open and a lower end seating the stopper when the float valve is closed.

14. The liquid dispenser of claim 13, wherein the float valve comprises an opening extending further downward than the stopper when the float valve is in the open position.

15. A liquid feed system, comprising:
a connector line in fluid communication with a pump;
a plurality of bottles; and
a plurality of liquid dispensers, each liquid dispenser comprising:
a cap receiving one of the plurality of bottles an inverted position;
a float valve positioned to extend into the received bottle, the float valve moving between an open position allowing liquid to enter from the bottle and a closed position when the bottle is depleted; and
a connector in fluid communication with the float valve;
wherein the connector line is in fluid communication with each of the dispensers in series, such that upon activation of the pump, fluid is drawn from the plurality bottles; and
wherein, when a liquid supply available in any one of the plurality of bottles is depleted, the float valve of the liquid dispenser receiving the depleted bottle closes to prevent further draw from the depleted bottle.

16. The liquid feed system of claim 15, each liquid dispenser further comprising legs extending below the connector to allow the liquid dispenser to stand when connected with an inverted bottle.

17. The liquid feed system of claim 15, wherein the cap of each liquid dispenser is threaded to match the threads of a bottle.

18. The liquid feed system of claim 15, wherein the float valve of each liquid dispenser comprises a housing, the housing having an upper end containing a stopper when the float valve is open and a lower end seating the stopper when the float valve is closed.

19. The liquid feed system of claim 15, wherein the float valve of each liquid dispenser comprises an opening extending further downward than the stopper when the float valve is in the open position.

20. The liquid feed system of claim 15, wherein the system is connected to the irrigation feed line of an endoscopic surgical device.
